(19) United States
(12) Patent Application Publication
(10) Pub. No.: US 2026/0225978 A1
(43) Pub. Date: Aug. 6, 2026

(54) METHOD FOR PREPARING RARE EARTH FLUORIDES

(71) Applicant: Iowa State University Research Foundation, Inc., Ames, IA (US)

(72) Inventors: Ikenna Cajetan Nlebedim, Ames, IA (US); Denis Prodius, Ames, IA (US); Anirudha Karati, Ames, IA (US); Tripta Parida, Ames, IA (US)

(73) Assignee: Iowa State University Research Foundation, Inc., Ames, IA (US)

(21) Appl. No.: 19/532,839

(22) Filed: Feb. 6, 2026

Related U.S. Application Data
(60) Provisional application No. 63/754,815, filed on Feb. 6, 2025.

(51) Int. Cl. C07C 21/18; C07B 59/00; C07C 19/08

(52) U.S. Cl. CPC C07C 21/18; C07B 59/002; C07C 19/08

(57) ABSTRACT

Embodiments of the present disclosure relate to a method of producing a rare earth trifluoride. In the method, a rare earth sulfate or oxalate or rare earth salt (halide) is reacted with a fluoride salt or metal rare earth fluoride to produce the rare earth trifluoride. The reaction is performed at a temperature of 100° C. or less and without using hydrofluoric acid and without producing hydrofluoric acid. Advantageously, the rare earth trifluoride does not evolve hydrofluoric acid when heated between room temperature and 1000° C. Additionally, the product of the reaction includes a major phase of the rare earth trifluoride, and at least 98 wt % of rare earth metal in the rare earth feedstock is converted to rare earth fluoride.

### CROSS-REFERENCE TO RELATED PATENT APPLICATIONS

This patent application claims the benefit of U.S. Provisional Patent Application No. 63/754,815, filed Feb. 6, 2025, the entire teachings and disclosure of which are incorporated herein by reference thereto.

### STATEMENT REGARDING FEDERALLY SPONSORED RESEARCH AND DEVELOPMENT

This invention was made with government support under DE-AC02-07CH11358 awarded by the Department of Energy. The government has certain rights in the invention.

### FIELD OF THE INVENTION

This invention generally relates to rare earth metal precursors and, in particular, to methods of preparing rare earth metal fluorides.

### BACKGROUND OF THE INVENTION

Rare earth elements (REEs) are used to make permanent magnets (PMs), and the demand for the latter is estimated to quadruple by 2050. PMs find use in several applications, such as, transportation, defense, medical, and clean energy. The environmental toxicity and high energy consumption associated with REE processing and refining are limiting steps in the supply chain. Hence several REEs are considered as critical materials both from short- and medium-term supply risk perspectives. Manufacturing of permanent magnets require REEs to be in metallic forms, but there are currently significant bottlenecks in REE metals production, particularly in the United States.

REE metals can be produced by molten salt electrolysis or metallothermic processes using rare earth halides (REX₃, where X: F, Cl) as one of the feedstocks. The Ames process is a metallothermic reduction that produces rare earth metals according to Equation 1:

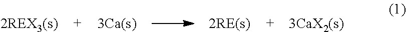

Among the rare earth halides, rare earth chlorides are air and moisture-sensitive and thus require handling and storage in an inert atmosphere. This significantly increases the cost and efficiency of using them as feedstock. Usually, rare earth fluorides are synthesized via wet and dry chemical methods and have greater air and moisture stability. In the wet processing method, rare earth salts can be reacted with aqueous hydrofluoric acid (HF) according to Equation 2, below, or ammonium bifluoride (NH₄·HF) to produce hydrated rare earth fluoride (REF₃·xH₂O). This is followed by the vaporization of water to obtain anhydrous rare earth fluoride. In Equation 2, Z can be anions like acetate, chloride, nitrate, etc.

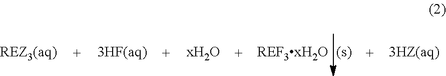

In the dry processing method, rare earth oxide is heat treated in a flow of dry HF gas to produce rare earth fluoride according to Equation 3:

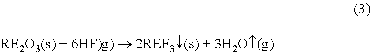

Because of several detrimental health effects of HF, aqueous ammonium bifluoride (NH₄F·HF) has been proposed. While NH₄F HF might minimize the challenges of handling the feedstock materials, it is not a true solution to the problem because HF is generated as a product of its reaction with the RE-salts according to Equation 4, below. NaF has also been proposed (see Equation 5, below) but it requires the use of aqueous acidic REX₃ solution with pH<3. The dissolution of NaF in acidic solutions can lead to the formation of HF as a by-product. The process cannot be used at higher pH because, at pH>3, the reaction leads to the formation of NaREF₄.

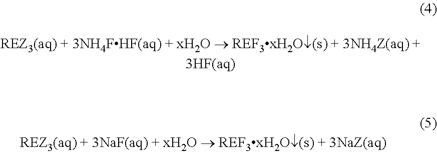

Since in commercial practice, both the electrolytic and metallothermic approaches require REF₃, Applicant has recognized that a safer and more environmentally friendly processing route is urgently needed.

### BRIEF SUMMARY OF THE INVENTION

Embodiments of the present disclosure relate to a method of obtaining rare earth fluoride without using hydrofluoric acid (HF). In one or more embodiments, the method is conducted at room temperature and at neutral pH conditions. In certain circumstances, such embodiments are expected to provide one or more of the following benefits. First, embodiments of the disclosed method are suitable for both mined and recycled feedstock materials. Second, HF is eliminated as a feedstock or product of the process, which improves operational safety. Third, REF₃ can be prepared at ambient conditions. Fourth, the process results in high phase purity (>95 wt. %) and >98 wt. % yield of product.

In a first aspect, embodiments of the present disclosure relate to a method of producing a rare earth trifluoride. In the method, a rare earth salt is reacted with a fluoride salt to produce the rare earth trifluoride. The method is performed at a temperature of 100° C. or less and without using hydrofluoric acid.

In a second aspect, embodiments of the present disclosure relate to the method of the first aspect in which the fluoride salt is at least one of ammonium fluoride, lithium fluoride, sodium fluoride, potassium fluoride, or a metal rare earth fluoride where the metal is at least one of lithium, sodium, potassium, or ammonium.

In a third aspect, embodiments of the present disclosure relate to the method of the first aspect in which the rare earth trifluoride comprises at least one of scandium, yttrium, lanthanum, cerium, praseodymium, neodymium, promethium, samarium, europium, gadolinium, terbium, dysprosium, holmium, erbium, thulium, ytterbium, or lutetium.

In a fourth aspect, embodiments of the present disclosure relate to the method of any of the first aspect to the third aspect in which the rare earth salt is a rare earth sulfate or oxalate.

In a fifth aspect, embodiments of the present disclosure relate to the method of any of the first aspect to the third aspect in which prior to reacting, the method further comprises preparing the rare earth sulfate or oxalate by reacting a rare earth salt with a sulfate or oxalate salt, or oxalic acid.

In a sixth aspect, embodiments of the present disclosure relate to the method of the fourth aspect or the fifth aspect in which the rare earth salt is produced by reacting at least one of a rare earth oxide, a rare earth hydroxide, a rare earth metal, or a rare earth carbonate with an acid.

In a seventh aspect, embodiments of the present disclosure relate to the method of the sixth aspect in which the acid is at least one of nitric acid (HNO₃), acetic acid (CH₃COOH), or a chlorine-containing acid (HCl or HClO₁₋₄).

In an eighth aspect, embodiments of the present disclosure relate to the method of the sixth aspect or the seventh aspect in which the rare earth oxide is REOₐ in which a is 1.5 for a trivalent rare earth oxide and 2 for a tetravalent rare earth oxide.

In a ninth aspect, embodiments of the present disclosure relate to the method of any of the sixth aspect to the eighth aspect in which the rare earth oxide is derived from mined ore, coal, coal-by-products, tailings, clays, and acid mine drainage.

In a tenth aspect, embodiments of the present disclosure relate to the method of any of the sixth aspect to the eighth aspect in which the rare earth oxide is derived from recycled waste.

In an eleventh aspect, embodiments of the present disclosure relate to the method of any of the fourth aspect to the tenth aspect in which the sulfate or oxalate salt comprises a cation selected from at least one of ammonium, lithium, sodium, or potassium.

In a twelfth aspect, embodiments of the present disclosure relate to the method of any of the first aspect to the eleventh aspect in which the rare earth fluoride is hydrated.

In a thirteenth aspect, embodiments of the present disclosure relate to the method of any of the first aspect to the twelfth aspect in which the rare earth fluoride does not evolve HF when heated between room temperature and 1000° C.

In a fourteenth aspect, embodiments of the present disclosure relate to a method of producing a rare earth trifluoride. In the method, a rare earth salt is reacted with a fluoride salt to produce the rare earth trifluoride. The rare earth salt comprises a sulfate or an oxalate, or the fluoride salt comprises a metal RE fluoride double salt.

In a fifteenth aspect, embodiments of the present disclosure relate to the method of the fourteenth aspect in which the method is performed at a temperature of 100° C. or less.

In a sixteenth aspect, embodiments of the present disclosure relate to the method of the fourteenth aspect or fifteenth aspect in which the method does not involve the use of hydrofluoric acid.

In a seventeenth aspect, embodiments of the present disclosure relate to the method of any of the fourteenth aspect to the sixteenth aspect in which the rare earth fluoride is a major phase.

In an eighteenth aspect, embodiments of the present disclosure relate to the method of any of the fourteenth aspect to the seventeenth aspect in which the rare earth trifluoride comprises at least one of scandium, yttrium, lanthanum, cerium, praseodymium, neodymium, promethium, samarium, europium, gadolinium, terbium, dysprosium, holmium, erbium, thulium, ytterbium, or lutetium.

In a nineteenth aspect, embodiments of present disclosure relate to the method of any of the fourteenth aspect to the eighteenth aspect in which the method further comprises reacting a rare earth oxide, hydroxide, metal, or carbonate with an acid to produce a first rare earth salt. The first rare earth salt is then with a sulfate, an oxalate, or oxalic acid to obtain the rare earth salt.

In a twentieth aspect, embodiments of the present disclosure relate to the method of the nineteenth aspect in which the acid is at least one of nitric acid (HNO₃), acetic acid (CH₃COOH), or a chlorine-containing acid selected from a group consisting of HCl, HClO₁₋₄, and combinations thereof.

In a twenty-first aspect, embodiments of the present disclosure relate to the method of any of the fourteenth aspect to the twentieth aspect in which, in the method, at least 98 wt % of rare earth content of a starting material is converted to rare earth trifluoride.

In a twenty-second aspect, embodiments of the present disclosure relate to the method of any of the fourteenth aspect to the twenty-first aspect in which a rare earth starting material used to produce the rare earth trifluoride is derived from at least one of mined ore, coal, coal by-products, tailings, clays, acid mine drainage, or recycled waste

In a twenty-third aspect, embodiments of the disclosure relate to a method in which a double salt of a rare earth element (RE) is reacted in an aqueous solution to produce an RE fluoride of the form REF₃ without using hydrofluoric acid and, preferably, without producing hydrofluoric acid.

In a twenty-fourth aspect, embodiments of the disclosure relate to the method of the twenty-third aspect in which the double salt comprises the RE, a cation, and an anion.

In a twenty-fifth aspect, embodiments of the disclosure relate to the method of the twenty-fourth aspect in which the cation comprises at least one of ammonium, lithium, sodium, or potassium.

In a twenty-sixth aspect, embodiments of the disclosure relate to the method of the twenty-fourth aspect in which the anion comprises at least one of sulfate, oxalate, or fluoride.

In a twenty-seventh aspect, embodiments of the disclosure relate to the method of the twenty-fourth aspect in which the aqueous solution comprises a fluoride salt.

In a twenty-eighth aspect, embodiments of the disclosure relate to the method of the twenty-seventh aspect in which the anion comprises sulfate.

In a twenty-ninth aspect, embodiments of the disclosure relate to the method of the twenty-fourth aspect in which the aqueous solution is boiling water.

In a thirtieth aspect, embodiments of the disclosure relate to the method of the twenty-ninth aspect in which the anion comprises oxalate.

In a thirty-first aspect, embodiments of the disclosure relate to the method of the twenty-fourth aspect in which the aqueous solution comprises an RE salt other than an RE fluoride, such as an RE chloride salt, an RE nitrate salt, or an RE acetate salt, amongst other possibilities.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a flow diagram of a method for producing rare earth fluorides using sodium fluoride, according to exemplary embodiments of the present disclosure;

FIG. 2 depicts a Rietveld refinement pattern of a rare earth fluoride obtained from sodium rare-earth sulfate and sodium fluoride reaction, according to an exemplary embodiment of the present disclosure;

FIG. 3 is a secondary electron micrograph demonstrating ~100 nm particle size of rare earth fluoride obtained from sodium rare-earth (didymium) sulfate and sodium fluoride reaction, according to an exemplary embodiment of the present disclosure;

FIG. 4 is a mass spectrometry profile of didymium fluoride demonstrating only water loss at temperatures in a range below 300° C. and no evolution of hydrofluoric acid, according to an exemplary embodiment of the present disclosure;

FIG. 5 is a powder X-ray diffraction pattern of NaNdF₄·xH₂O produced using oxalate precursors, according to an exemplary embodiment of the present disclosure;

FIGS. 6A and 6B are differential scanning calorimetry and thermogravimetric analysis curves and mass spectrometry profiles, respectively, for the NaNdF₄·xH₂O powder as heated up to 600° C., according to an exemplary embodiment of the present disclosure;

FIG. 7 is a powder X-ray diffraction pattern of NdF₃ following hot water-washing, according to an exemplary embodiment of the present disclosure;

FIGS. 8A and 8B are thermogravimetric analysis curves and mass spectrometry profiles, respectively, for NdF₃ powder as heated up to 600° C., according to an exemplary embodiment of the present disclosure;

FIG. 9 is a powder X-ray diffraction pattern of NaDiF₄·xH₂O produced using oxalate precursors, according to an exemplary embodiment of the present disclosure;

FIG. 10 is a powder X-ray diffraction pattern of DiF₃ following hot water-washing, according to an exemplary embodiment of the present disclosure; and

FIG. 11 provides X-ray diffraction patterns for NdF₃ prepared from NaNdF₄ starting material prepared at room temperature and at 70° C., according to exemplary embodiments of the present disclosure.

While the invention will be described in connection with certain preferred embodiments, there is no intent to limit it to those embodiments. On the contrary, the intent is to cover all alternatives, modifications, and equivalents as included within the spirit and scope of the invention as defined by the appended claims.

### DETAILED DESCRIPTION OF THE INVENTION

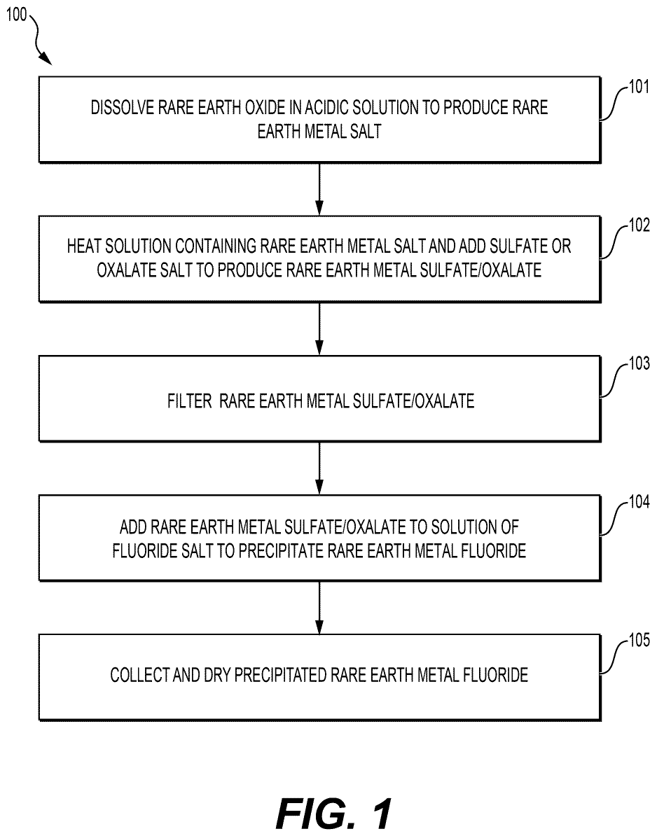

FIG. 1 depicts a flow diagram of a method 100 for preparing rare earth (RE) metal fluorides according to embodiments of the present disclosure. According to embodiments of the method 100, the starting material containing the metal is a rare earth oxide (REOₐ). In the first step 101 of the method 100, the rare earth oxide is dissolved in an acidic solution to produce a hydrated rare earth salt according to Equation 6:

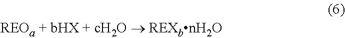

in which X is the anion of the acid, a is typically in a range from 1.5 to 2, b is typically in a range from 3 to 4, n is a number of crystallization water molecules, and c is the number of water molecules necessary for stoichiometric balance.

In one or more embodiments, the acidic solution comprises from 1% to 68% acid in water. In one or more embodiments, the acid is at least one of nitric acid (HNO₃), acetic acid (CH₃COOH), or a chlorine-containing acid (HCl or HClO₁₋₄). In one or more embodiments, the rare earth is at least one of scandium, yttrium, lanthanum, cerium, praseodymium, neodymium, promethium, samarium, europium, gadolinium, terbium, dysprosium, holmium, erbium, thulium, ytterbium, or lutetium. For example, the rare earth may be combination of neodymium (Nd) and praseodymium (Pr), which is known as didymium (Di), and/or other rare earth combinations.

In one or more embodiments, the first step 101 of the method is performed at room temperature, and the rare earth oxide is stirred in the aqueous acidic solution.

In a second step 102 of the method 100, the hydrated rare earth salt is provided in an aqueous solution prepared to exclude acidic media and heated to a temperature in a range of 40° C. to 100° C. Further, in the second step, sulfate or oxalate salt is added in small proportions, preferably under constant stirring. In one or more embodiments, the sulfate or oxalate salt comprises a cation selected from ammonium, lithium, sodium, or potassium. The hydrated rare earth salt and sulfate or oxalate salt react according to Equation 7, below. In Equation 7, a sulfate is used to illustrate the reaction, but the sulfate could be replaced with oxalate (C₂O₄²⁻). In particular, with respect to Equation 7, the sulfate salt is sodium sulfate (Na₂SO₄), and the reaction precipitates sodium rare earth double sulfate:

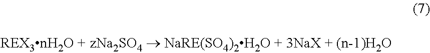

in which z represents the excess amount of sodium sulfate necessary to convert quantitatively rare earth (RE) to the rare earth double sulfate salt.

In a third step 103 of the method 100, the solution is filtered according to standard processes to collect the rare earth double sulfate/oxalate salt, and the rare earth double sulfate/oxalate salt may be dried. In one or more embodiments, the drying may be performed in ambient air.

In a fourth step 104 of the method 100, the rare earth double sulfate/oxalate salt precipitate is added to a solution of fluoride salt (such as lithium fluoride, sodium fluoride, and/or potassium fluoride), and the mixture is stirred to react the rare earth double sulfate/oxalate and fluoride salt to produce hydrated rare earth fluoride. In one or more embodiments, the mixture is stirred at room temperature. Equation 8, below, describes the reaction in the fourth step 104 of the method 100. As with the previous Equation, sulfate is used for illustrative purposes in Equation 8, but the sulfate could be replaced with oxalate.

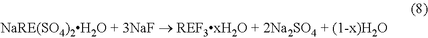

The hydrated rare earth fluoride precipitates from the mixture, and in a fifth step 105, the precipitated hydrated rare earth fluoride is collected and dried. In one or more embodiments, the hydrated rare earth fluoride can be dehydrated by heating the hydrated rare earth fluoride up to 600° C. Additionally or alternatively, the hydrated rare earth fluoride may be washed in boiling water before drying.

In another embodiment, the precursor for the method is a rare earth double salt, in particular a rare earth fluoride double salt of the form MₙREF_{4y} in which Mis a cation selected from ammonium, lithium, sodium, or potassium and in which n=y=1 to 4. The rare earth double salt is reacted in an aqueous solution. In the embodiments described above, the rare earth double salt is reacted in an aqueous solution where the aqueous solution comprises a fluoride salt or is hot water. However, in one or more embodiments where the precursor is a rare earth fluoride double salt, the aqueous solution may comprise a rare earth salt (e.g., having the form of REX₃) in which the salt is not a fluoride salt. In one or more embodiments, the salt is a chloride, a nitrate, or an acetate, amongst other possibilities. In such embodiments, the rare earth fluoride double salt of the form MₙREF_{4y} will react with REX₃ to form REF₃, which is the preferred form of rare earth fluoride used in industry.

The rare earth fluoride produced according to the present disclosure can then be utilized in various related applications. Advantageously, producing the rare earth fluorides according to the present disclosure method avoids the use of hazardous hydrofluoric acid and does not produce hydrofluoric acid as a byproduct.

### EXPERIMENTAL EXAMPLES

### Example 1

Didymium (Di) fluoride was synthesized from sodium Di double sulfate salt as described below. The Di comprised Nd/Pr (75/25).

A slight excess of Di oxide was dissolved in 50% HNO₃ and stirred at room temperature to produce Di(NO₃)₃·6H₂O according to Equation 9:

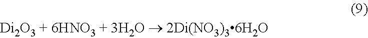

The aqueous rare earth nitrate solution (prepared to exclude acidic media) was heated to 80° C., and Na₂SO₄ was added in small portions under constant stirring as shown in Equation 10, below. The solution was filtered, and the NaDi(SO₄)₂·H₂O precipitate was collected and dried in ambient air.

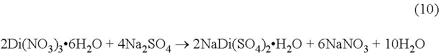

To an aqueous solution of sodium fluoride, NaDi(SO₄)₂·H₂O was added, and the contents were stirred at room temperature and the reaction proceeded according to Equation 11, below. The solution was filtered, and the DiF₃ xH₂O precipitate was collected and dried.

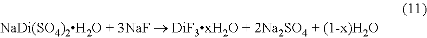

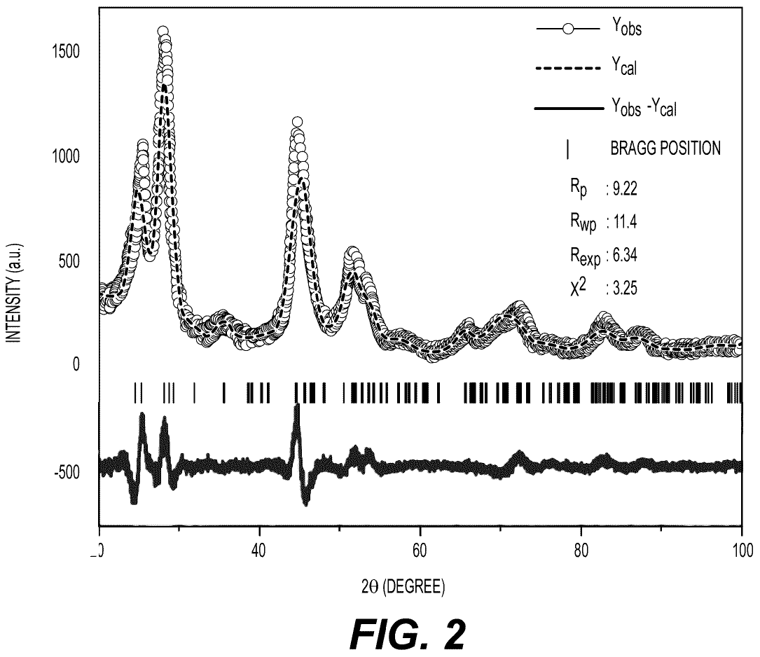

The collected DiF₃·xH₂O was analyzed to confirm the presence of a DiF₃-based trifluoride phase. In particular, a powder x-ray diffraction (XRD) pattern was recorded, and Rietveld refinement analysis was performed to confirm the presence of DiF₃-based trifluoride. FIG. 2 provides the Rietveld refinement pattern of the rare earth fluoride obtained according to this experimental example. As can be seen in FIG. 2, the peak broadening in the XRD pattern indicated the nanocrystalline nature of the sample.

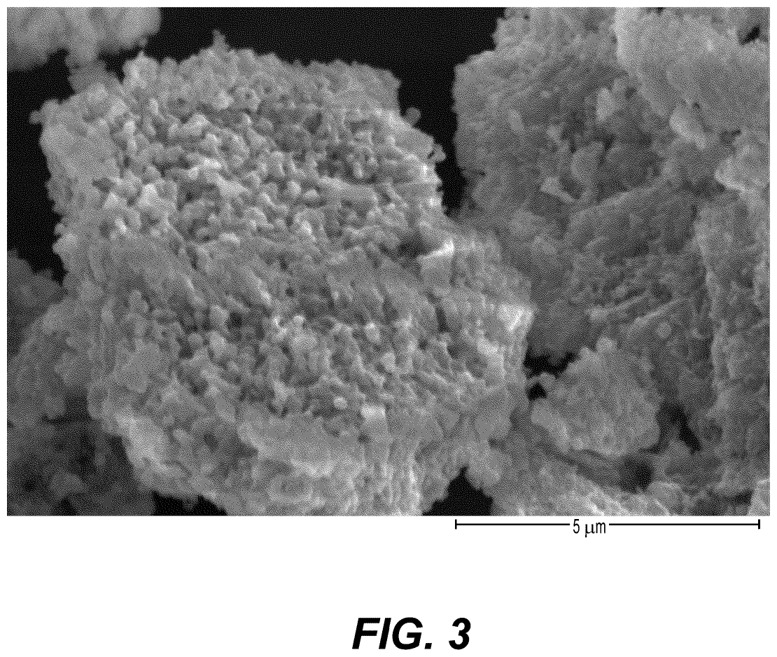

Using a scanning electron microscope to collect a secondary electron micrograph, shown in FIG. 3, it was determined that the rare earth fluoride produced in the experimental example had a particle size of ~100 nm.

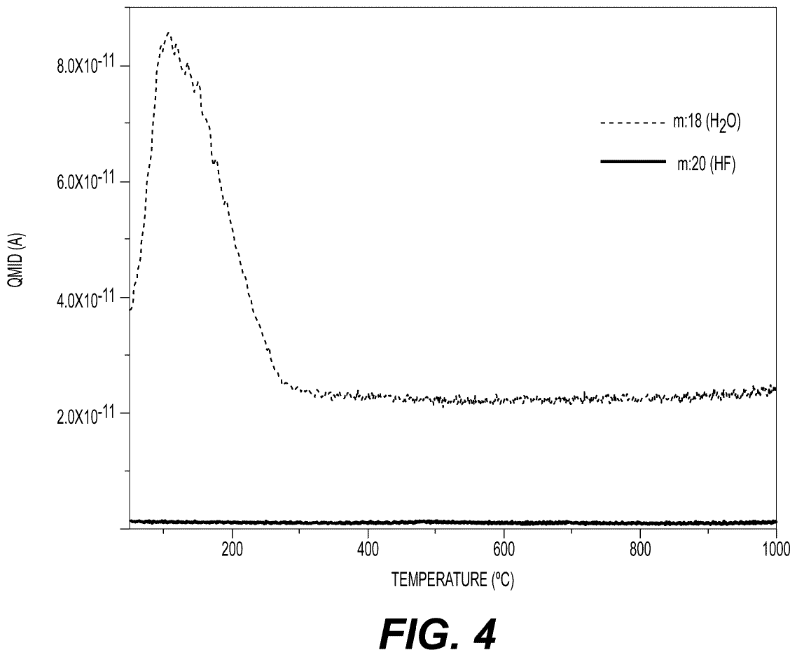

In order to determine whether any hydrofluoric acid was produced or would evolve from reactions involving the rare earth fluoride, thermogravimetric analysis (TGA) was performed. TGA demonstrated that the rare earth fluoride sample exhibited a mass loss of about 4% at temperatures below 300° C. A mass spectrometry analysis was recorded to understand the evolved species during the mass loss event, and as can be seen from FIG. 4, it was concluded that water was the sole chemical that evolved between 30° C. and 300° C. No peak corresponding to hydrofluoric acid (HF) release was observed.

### Example 2

A stoichiometric amount of neodymium oxide (Nd₂O₃) was added to a hydrochloric acid (HCl) solution and stirred to obtain a clear solution. The reaction between the neodymium oxide and hydrochloric acid proceeded according to the following reaction:

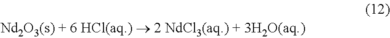

As can be seen from Equation 12, the reaction produced an aqueous solution containing neodymium(III) chloride (NdCl₃).

A stoichiometric amount of ammonium oxalate monohydrate was dissolved in distilled water to prepare a clear aqueous solution of ammonium oxalate ((NH₄)₂C₂O₄). The aqueous solution of ammonium oxalate was mixed with the aqueous solution of neodymium(III) chloride according to Equation 13, below.

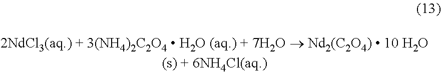

As can be seen from Equation 13, the neodymium(III) chloride reacted with the ammonium oxalate to produce hydrated neodymium(III) oxalate (Nd₂(C₂O₄)₃·10H₂O) and ammonium chloride (NH₄Cl). The hydrated neodymium(III) oxalate precipitated as a solid from solution and was separated.

Sodium fluoride (NaF) was dissolved in water. Once fully dissolved, the solid hydrated neodymium(III) oxalate was added to the sodium fluoride solution, and the mixture was stirred, resulting in the following reaction:

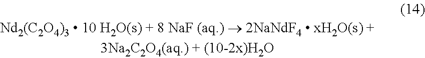

As can be seen from Equation 14, the hydrated neodymium(III) oxalate reacted with the sodium fluoride to produce a solid mixed metal salt of hydrated sodium neodymium fluoride, which precipitated from the solution. In particular, the sodium neodymium(III) oxalate remained solid within the mixture and underwent an ion-exchange reaction to replace the oxalate groups with fluorides from the sodium fluoride in solution. The solution was then filtered, and the resulting powder was dried in an oven, which dehydrated the sodium neodymium fluoride.

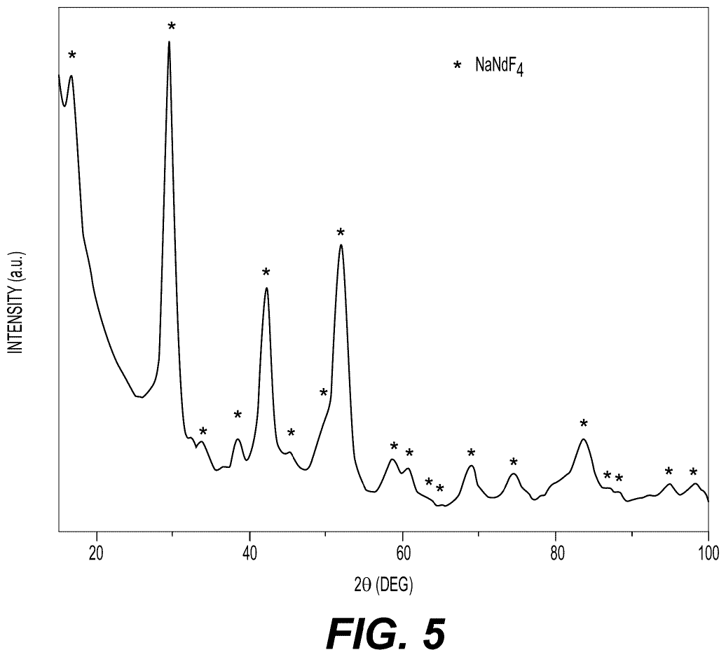

In order to confirm composition of the powder, the dried powder was characterized using powder x-ray diffraction (PXRD). The PXRD spectrum is shown in FIG. 5, and the spectrum includes many peaks consistent with the presence of sodium neodymium fluoride.

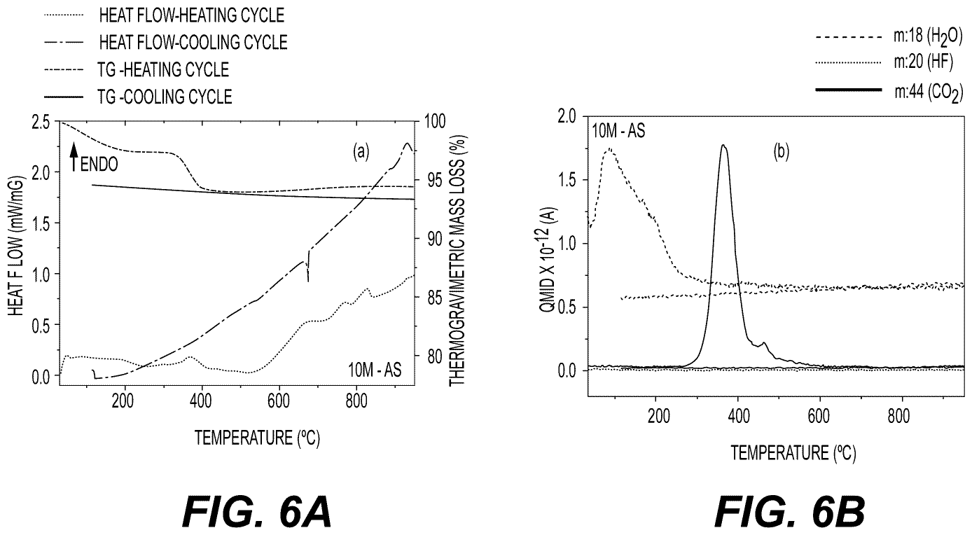

Differential scanning calorimetry (DSC), thermogravimetric analysis (TGA), and mass spectrometry (MS) were performed on a sample of the dried powder. The TGA and DSC curves are shown in FIG. 6A, and the MS profile is shown in FIG. 6B. The TGA curve of FIG. 6A shows a 6% mass loss below 600° C., attributed to the release of water and carbon dioxide. With respect to the MS profile of FIG. 6B, no signal corresponding to HF evolution was detected.

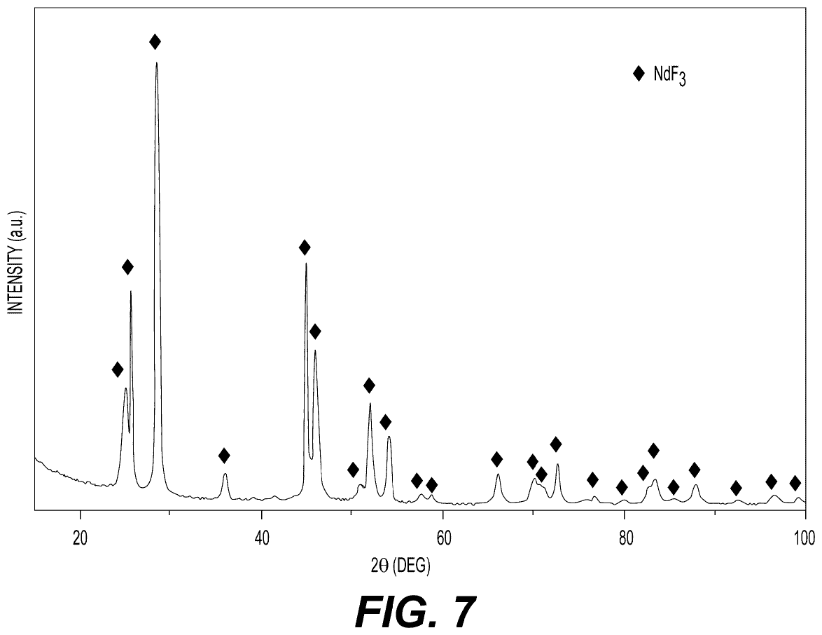

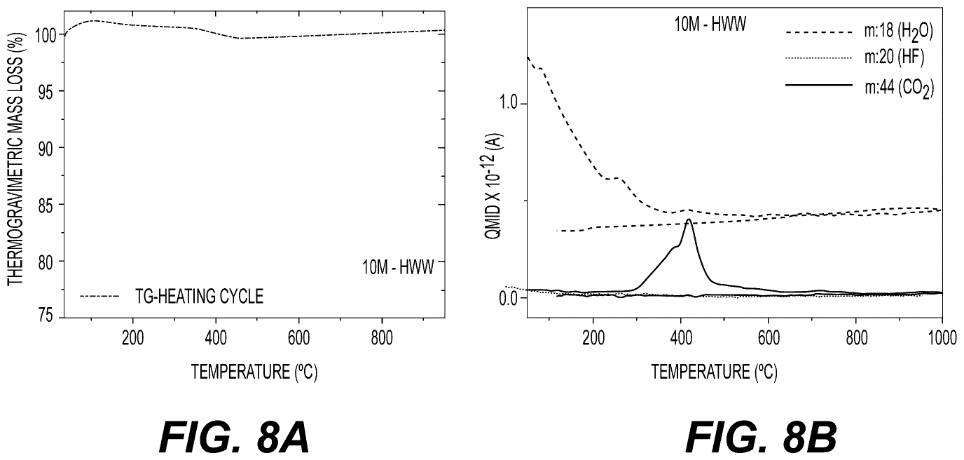

Deionized water was heated to boiling (100° C.), and the NaNdF₄·xH₂O was added to the boiling water. The mixture was stirred, then filtered and dried in an oven. The XRD pattern of the hot water-washed (HWW) dried powder confirmed the formation of single-phase NdF₃, as shown in FIG. 7. DSC/TGA and MS were carried out on a sample of this powder as well. The TGA and DSC curves are shown in FIG. 8A, and the MS profile is shown in FIG. 8B. TGA analysis in FIG. 8A indicates a 1.5% mass loss below 600° C., attributed to the release of volatile components. In the MS profile of FIG. 8B, no HF-related peaks were observed.

### Example 3

A stoichiometric amount of didymium oxide (Di₂O₃) was added to a hydrochloric acid (HCl) solution and stirred to obtain a clear solution. The reaction between the didymium oxide and hydrochloric acid proceeded according to the following reaction:

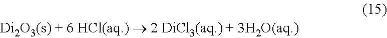

As can be seen from Equation 15, the reaction produced an aqueous solution containing didymium(III) chloride (DiCl₃).

A stoichiometric amount of ammonium oxalate monohydrate was dissolved in distilled water to prepare a clear aqueous solution of ammonium oxalate ((NH₄)₂C₂O₄). The aqueous solution of ammonium oxalate was mixed with the aqueous solution of didymium(III) chloride according to Equation 16, below.

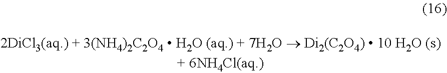

To synthesize NaDiF₄·xH₂O, a slight excess of sodium fluoride (NaF) was dissolved in water. Once fully dissolved, Di₂(C₂O₄)₃·10 H₂O was added, and the mixture was stirred. The equation corresponding to the above reaction is as follows:

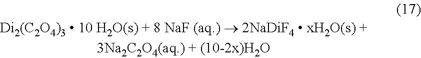

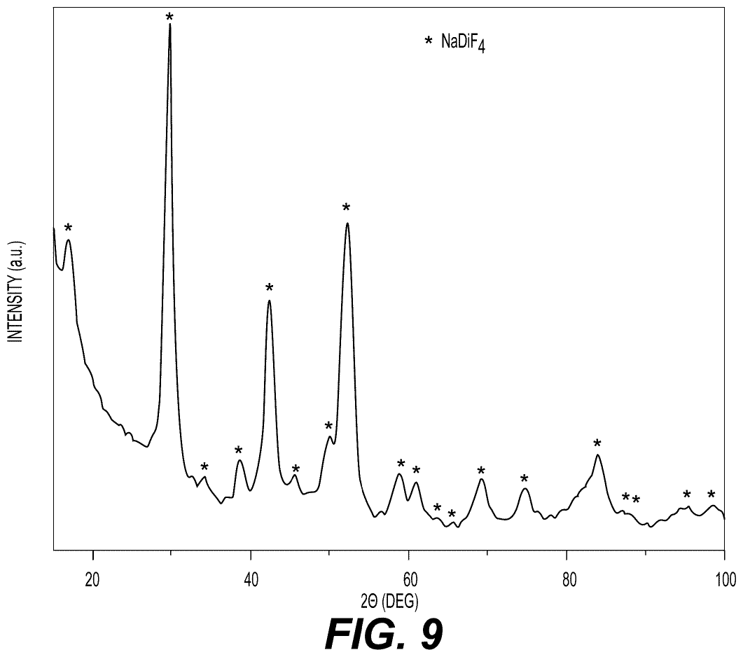

The solution was filtered, and the powder was dried. PXRD analysis of the dried powder confirmed the formation of crystalline NaDiF₄, as shown in FIG. 9.

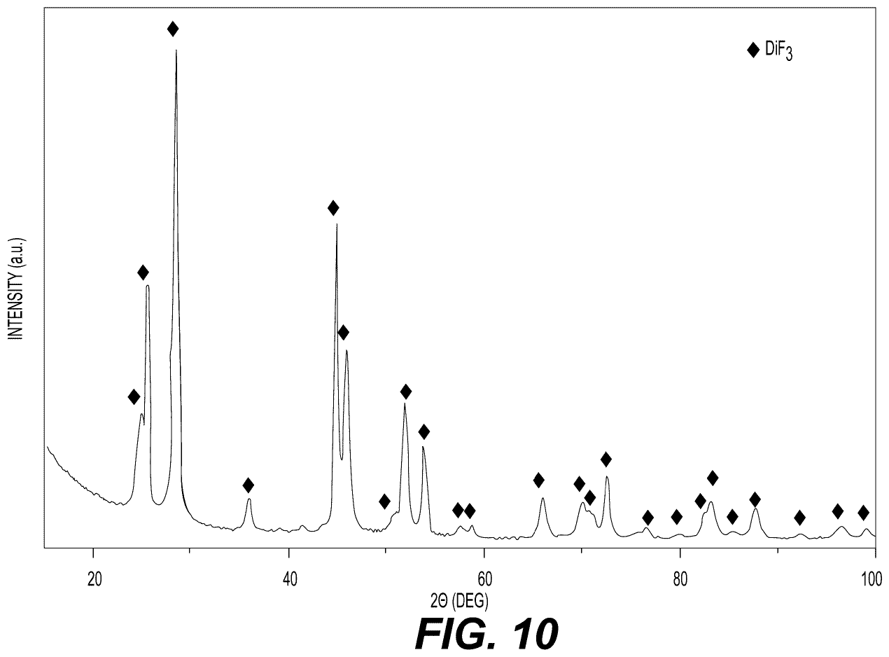

The as-synthesized NaDiF₄·x H₂O was added to the boiling water and stirred. After the reaction, the mixture was filtered, and the solid product was dried in an oven. PXRD analysis of the hot water-washed (HWW) powder revealed the formation of pure DiF₃ phase, as illustrated in FIG. 10.

### Example 4

According to this example, neodymium(III) fluoride (NdF₃) was prepared from NaNdF₄. First, a solution of NdX₃ rare earth salt (in particular, NdCl₃) was diluted with deionized water, after which a metal or ammonium rare earth fluoride salt (NaNdF₄) was introduced. The mixture was stirred under room temperature conditions (~25° C.) to generate an NdF₃ product according to Equation 18, below.

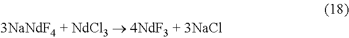

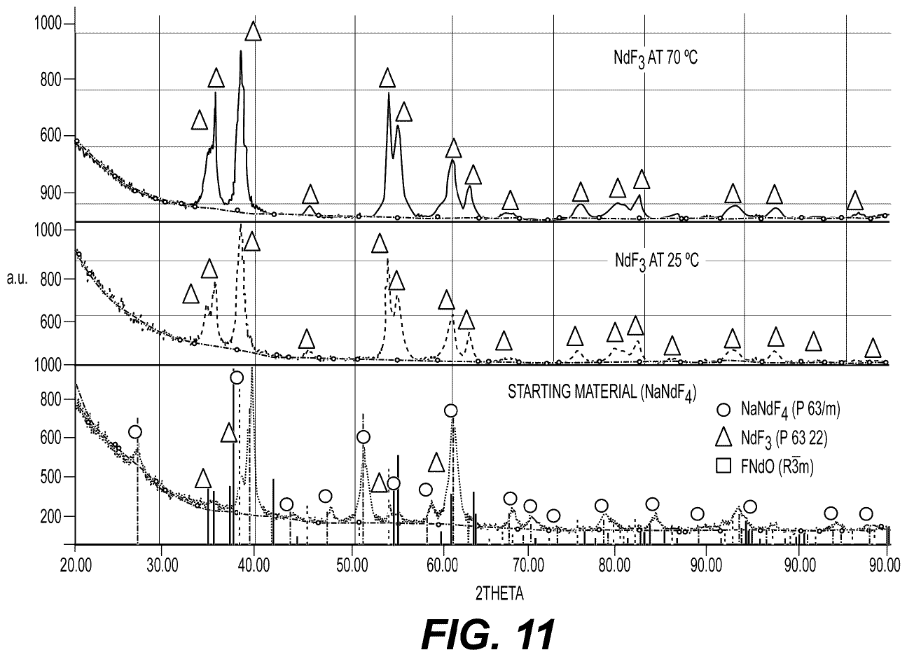

A corresponding reaction was performed under elevated-temperature conditions (~70° C.) to obtain a second NdF₃ sample. The resulting solids were isolated, dried, and analyzed using X-ray diffraction and inductively coupled plasma methods. The X-ray diffraction patterns are shown in FIG. 11.

The reactions resulted in NdF₃ product with ≥95% yield. Applicant believes that even higher NdF₃ phase purity can be achieved through further processing.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of producing a rare earth trifluoride, comprising:
reacting a rare earth salt with a fluoride salt to produce the rare earth trifluoride;
wherein the method is performed at a temperature of 100° C. or less and without using hydrofluoric acid.

2. The method of claim 1, wherein the fluoride salt comprises at least one of ammonium fluoride, lithium fluoride, sodium fluoride, potassium fluoride, or a metal rare earth fluoride where the metal is at least one of lithium, sodium, potassium, or ammonium.

3. The method of claim 1, wherein the rare earth trifluoride comprises at least one of scandium, yttrium, lanthanum, cerium, praseodymium, neodymium, promethium, samarium, europium, gadolinium, terbium, dysprosium, holmium, erbium, thulium, ytterbium, or lutetium.

4. The method of claim 1, wherein the rare earth salt is a rare earth sulfate or oxalate.

5. The method of claim 4, wherein prior to reacting, the method further comprises preparing the rare earth sulfate or oxalate by reacting a rare earth compound with a sulfate or oxalate salt or with an oxalic acid.

6. The method of claim 4, wherein the rare earth salt is produced by reacting at least one of a rare earth oxide, a rare earth hydroxide, a rare earth metal, or a rare earth carbonate with an acid.

7. The method of claim 6, wherein the acid is at least one of nitric acid (HNO₃), acetic acid (CH₃COOH), or a chlorine-containing acid selected from a group consisting of HCl, HClO₁₋₄, and combinations thereof.

8. The method of claim 6, wherein the rare earth oxide is REOₐ in which a is 1.5 for a trivalent rare earth oxide and 2 for a tetravalent rare earth oxide.

9. The method of claim 6, wherein the rare earth oxide is derived from at least one of mined ore, coal, coal by-products, tailings, clays, or acid mine drainage.

10. The method of claim 6, wherein the rare earth oxide is derived from recycled waste.

11. The method of claim 4, wherein the sulfate or oxalate salt comprises a cation selected from at least one of ammonium, lithium, sodium, or potassium.

12. The method of claim 1, wherein the rare earth trifluoride is hydrated.

13. The method of claim 1, wherein the rare earth trifluoride does not evolve HF when heated between room temperature and 1000° C.

14. A method of producing a rare earth trifluoride, comprising:
reacting a rare earth salt with a fluoride salt to produce the rare earth trifluoride;
wherein (i) the rare earth salt comprises a sulfate or an oxalate or (ii) the fluoride salt comprises a metal RE fluoride double salt.

15. The method of claim 14, wherein the method is performed at a temperature of 100° C. or less.

16. The method of claim 14, wherein the method does not involve use of hydrofluoric acid and the method does not produce hydrofluoric acid.

17. The method of claim 14, wherein, after reacting the rare earth salt with the fluoride salt, the rare earth trifluoride is present as a major phase.

18. The method of claim 14, wherein the rare earth trifluoride comprises at least one of scandium, yttrium, lanthanum, cerium, praseodymium, neodymium, promethium, samarium, europium, gadolinium, terbium, dysprosium, holmium, erbium, thulium, ytterbium, or lutetium.

19. The method of claim 14, wherein the method further comprises reacting a rare earth oxide, hydroxide, metal, or carbonate with an acid to produce a first rare earth salt; and
reacting the first rare earth salt with a sulfate, an oxalate, or oxalic acid to obtain the rare earth salt.

20. The method of claim 19, wherein the acid is at least one of nitric acid (HNO₃), acetic acid (CH₃COOH), or a chlorine-containing acid selected from a group consisting of HCl, HClO₁₋₄, and combinations thereof.

21. The method of claim 19, wherein, in the method, at least 98 wt % of rare earth content in the rare earth oxide, hydroxide, metal, or carbonate is converted to rare earth trifluoride.

22. The method of claim 19, wherein the rare earth oxide, hydroxide, metal, or carbonate is derived from at least one of mined ore, coal, coal by-products, tailings, clays, acid mine drainage, or recycled waste.

23. A method comprising:
reacting a double salt of a rare earth element (RE) in an aqueous solution to produce an RE fluoride having a form of REF₃ without using hydrofluoric acid.

24. The method of claim 23, wherein the double salt comprises the RE, a cation, and an anion.

25. The method of claim 24, wherein the cation comprises at least one of ammonium, lithium, sodium, or potassium.

26. The method of claim 24, wherein the anion comprises at least one of sulfate, oxalate, or fluoride.

27. The method of claim 24, wherein the aqueous solution comprises a fluoride salt.

28. The method of claim 27, wherein the anion comprises sulfate.

29. The method of claim 24, wherein the aqueous solution is boiling water.

30. The method of claim 29, wherein the anion comprises oxalate.

31. The method of claim 24, wherein the aqueous solution comprises a RE salt other than an RE fluoride.

32. The method of claim 31, wherein the RE salt comprises an RE chloride salt, an RE nitrate salt, or an RE acetate salt.
